(19) United States
(12) Patent Application Publication
(10) Pub. No.: US 2021/0290882 A1
(43) Pub. Date: Sep. 23, 2021

(54) NASAL-PRONG AND NASAL-MASK CANNULA AND GAS TANK SUPPLY SYSTEM

(71) Applicants: Elaine CONCKLIN, Wilmington, DE (US); Richard Hoffman, Hockessin, DE (US)

(72) Inventors: Elaine CONCKLIN, Wilmington, DE (US); Richard Hoffman, Hockessin, DE (US)

(21) Appl. No.: 17/339,584

(22) Filed: Jun. 4, 2021

Related U.S. Application Data
(63) Continuation-in-part of application No. PCT/US2019/067640, filed on Dec. 19, 2019.
(60) Provisional application No. 62/782,759, filed on Dec. 20, 2018.

(51) Int. Cl. A61M 16/06

(52) U.S. Cl. CPC A61M 16/0672; A61M 2205/6081; A61M 2202/0208; A61M 16/0683

(57) ABSTRACT

A nasal prong or nasal mask cannula system which has at least one of the following features: to enhance the cannula tubing at the port base of the oxygen source to minimize “crimping” and diminished oxygen delivery; to design a cannula that will expand in length as needed and shorten as needed without loss of pressure (PSI) of oxygen from start point of oxygen source to patient; to provide a device such as a clasp or clip attached to the cannula which clasp or clip may be attached to the patient's clothing to relieve pressure or weight exerted by the cannula tubing on the patient's ears and keep the cannula close to the patient's body; to provide storage means for storing extra length of tubing; to provide a warning device to alert the patient when oxygen levels or oxygen flow rate are low or that the battery energizing the warning device is low; to provide a timing device to alert the patient to change the cannula after a prescribed period of time; to provide means for stylizing or personalizing the cannula system; to provide an oxygen supply system with a regulator which includes an external outlet or port that includes a pivot of at least 180° and optimally 360°.

### RELATED APPLICATIONS

This application is a continuation in part of PCT/US2019/067640 filed Dec. 19, 2019. PCT/US2019/067640 claims benefit to U.S. Provisional Application Ser. No. 62/782,759 filed Dec. 20, 2018. Both applications are incorporated by reference in their entirety.

### BACKGROUND OF THE INVENTION

This application relates generally to the field of cannulas used with nasal prongs or nasal masks, regulators, and gas tanks used to deliver oxygen or other gases from a tank supply and regulator to a patient through the nostrils, and more particularly relates to improving the use of the cannula and tank system.

Nasal prong cannulas for supplying oxygen or other gases to patients are well known in the medical field and are typically comprised of an elongated tubular main body having a pair of tubular nasal or nostril prongs extending substantially perpendicularly from the main body, the combination often being referred to as a nose piece. Patients with more severe symptoms of continuous positive airway pressure (“CPAP”) or bilevel positive airway pressure (BiPAP or BPAP) may need to wear a mask that covers the nose and mouth to allow for more oxygen flow than with their cannula tubing. The air pressure keeps the throat muscles from collapsing and reducing obstructions by acting as a splint. Both CPAP and BiPAP machines allow patients to breathe easily and regularly throughout the night. The tubular main body is open on both ends and connects with a regulator attached to a gas supply tank or cannula tubing such that gas, for example oxygen, may be delivered by a compressed-gas tank, a compressor, or a pump, through a regulator connected to one or both gas supply tubes into the tubular main body of the nasal cannula and then through the nostril prongs to be dispelled into the nasal passages of the patient.

To secure the device to the patient, the nose piece of the device is positioned below the nose with the nasal prongs inserted within the nostrils of the patient's nose. Below the nose, the tube separates into two tubes which wrap around the patient's ears and descending under the chin at which point the two tubes join to become one gas supply tube, joined together by a sliding collar such that the tubes can be tightened to maintain a snug fit of the nasal prongs to the patient's face. A patient with a nasal mask, instead of nasal prongs, will position the mask over the bridge of their nose and under their chin.

There are problems associated with the use of cannulas, regulators, and the supply tank. Crimping of the cannula can occur, for example, at the location where the cannula is connected to the regulator attached to an oxygen tank, concentrator, or other oxygen supply device, which restricts the flow of oxygen. In addition, the cannula tubing must be of sufficient length to allow the patient to move away from the oxygen tank or supply, but the additional tubing length can be cumbersome or get in the way of the patient's daily activities. In addition, when placed over the ears, the tubing can impose too much tension or weight on the patient's ears so as to cause discomfort. Typically, a regulator is attached to the oxygen supply. With most oxygen supplies, the regulator includes no warning device to alert the patient that the oxygen levels are nearing the critical level of zero liters or when the flow rate of gas is below the prescribed flow rate. Oxygen saturation (SaO₂) is a measurement of the percentage of how much hemoglobin is saturated with oxygen. Oxygen is transported in the blood in two ways: oxygen dissolved in blood plasma (pO₂) and oxygen bound to hemoglobin (SaO₂). Patients thus can be placed in a detrimental medical position of having too low SaO₂ saturation levels and not being aware that they need to replace their oxygen supply tank or battery to continue proper oxygen flow that is necessary for proper physiological SaO₂ levels. In addition, with most oxygen supplies the regulator does not include a timing device or mechanism to alert the patient that it is time to replace the cannula so as to avoid the deleterious effects of the buildup of bacteria or the deterioration or hardening of the tubing.

U.S. Pat. No. 10,034,995 which is incorporated by reference, discloses retractable cannula tube used for CPAP.

Nasal prong and nasal mask cannulas and their associated regulators and oxygen tanks all look virtually the same. In addition, patients would like to stylize and personalize their equipment which would also differentiate it from the equipment of others.

The gas supply system typically comprises a metal tank filled with gas, such as oxygen, a regulator connected to the tank indicating the flow rate of the gas, and a cannula with nasal prongs or nasal mask connected to the regulator.

Patients typically use several metal gas tanks per day and metal tanks can be difficult to handle and carry and create a hazardous situation when multiple metal gas tanks are stacked together, metal to metal, for storage or transport. The awkward style and bulkiness of the tanks is a barrier for patients to accept their use and comply with their medically prescribed oxygen needs.

### A SUMMARY OF THE INVENTION

It is an object of the invention to provide a system to improve the structure and design of cannulas, CPAP and CBiAP and the gas supply system to which they are connected.

Another object of the invention is to enhance the cannula tubing at the port base of the regulator attached to an oxygen source to minimize “crimping” and diminished oxygen delivery.

Another object of the invention is to design a cannula that will expand in length as needed and shorten as needed without loss of pressure (PSI) of oxygen from start point of oxygen source to the patient.

Another object of the invention is to provide a device, such as a clasp, clip, or hook which can be attached to the cannula tubing or other type of tubing and can also be attached to the patient's clothing so as to relieve the tension or weight placed on the ears by the cannula and to keep the cannula close to the patient's body to, among other things, improve ease of the person's movement and to reduce the risk of tripping on the tubing.

Another object of the invention is to provide a storage means for holding extra cannula length without crimping the cannula for portable oxygen cloth carry cases, wheelchair oxygen cloth carry cases, and oxygen constant/pulse flow tanks. For example, a slip-on “sock” or “cap” or “glove” or “sleeve” may be fitted over or around the oxygen tank which sock, cap or glove may include a Velcro® storage means made by Velcro out of the United Kingdom or a similar functioning product, which is a hook-and-loop fastener, flap, clasp, hook, or other fastening means to hold or fasten extra length of cannula tubing. These items may be personalized by or for the patient.

Another object of the invention is to provide a covering or device, such as a “sock” or “cap,” or “sleeve,” or “glove,” around the gas tank such that the tank is easier to handle and when multiple tanks are stacked next to each other the metal of one tank is not directly in contact with the metal of another tank. The covering or device may be open or closed at the bottom of the tank.

Another object of the invention is to provide one or more “pockets” or “pouches” on the aforementioned covering around the metal gas tank which can be used to store a patient's personal items including but not limited to a cell phone, wallet, keys, identification, or other items.

Another object of the invention is to provide a “shoulder strap,” a “handle” or “grip” on the aforementioned covering around the metal gas tank which can be used to carry the tank.

Another object of the invention is to provide a sensor, in the regulator, in the nasal prongs or mask or elsewhere, that monitors the gas supply in the tank and provides an alert to the patient when the gas supply is low or the flow rate of gas is above or below the prescribed flow rate.

Another object of the invention is to provide a bar code, tab release, or sensor in the cannula at or near the end that is connected to the regulator, which may be scanned or activated when the cannula is first put in use and which activates a timer for a prescribed time period, such as two weeks duration, and provides a color change or an audio, visual, or vibration alert to the patient at the end of the prescribed time period when it is time to replace the cannula.

Another object of the invention is to provide a warning sensor or other device, which will produce a visual cue, sound, vibration, or other alert transmitted to the patient or to a wireless device such as a smartphone or smartwatch to alert the patient that oxygen PSI levels in the tank or other oxygen supply is low or the flow rate of gas is above or below the prescribed flow rate and to alert the patient when the battery energizing the warning device is low. Stated differently, a smartphone, Apple watch, or Fitbit could be used as a monitoring device for oxygen levels, oxygen flow rate, and battery level.

Another object of the invention is to provide a timing function, with an interface with a wireless device such as a smartphone or smartwatch, to alert the patient after a prescribed time period, such as a two week time period has transpired since the first use of the cannula, so the patient knows it is time to replace the cannula so as to avoid hardening or the deleterious effects of bacteria buildup that may occur with extended use of the cannula and possible deterioration of the tubing.

Another object of this invention to provide an improved nasal prong or nasal mask cannula and oxygen tank supply system that includes a means of differentiating a patient's equipment from others and permits the patient to stylize his or her equipment.

Another object of the invention is to provide an improved regulator which includes an outlet, port, or coupling piece for connection to the gas source that incorporates a swivel pivot capable of rotation of at least 180° and optimally 360° in such a way as to prevent crimping of the cannula at the connection site.

Throughout the application patient is mentioned and patient, also includes person even if not specifically mentioned. In addition, throughout the application cannula tubing is mentioned and cannula tubing also includes other conventional tubing if not specifically mentioned.

### A BRIEF DESCRIPTION OF THE FIGURES

FIGS. 1 and 2 illustrate a cannula tube.

FIG. 3A illustrates the prior art of a crimped cannula tube.

FIG. 3B illustrates a cannula tube according to the invention.

FIG. 4 illustrates an embodiment with a tube-like structure with an external spring according to the invention as shown in FIG. 3B.

FIG. 5A illustrates Clasps with 360 degree pivot annotated for reference.

FIG. 5B illustrates the use of two integrated clasps rather than the use of a 360 degree pivot.

FIGS. 6 and 7 illustrates a tank system to hold the extra cannula tube according to the invention. The system shows an O₂ tank with sock and the Velcro® flap to hold cannula. The sock could also fit over top of the tank to avoid slippage when flap is used.

FIGS. 8 and 9 illustrates another embodiment to hold the extra cannula tube. The hook on tank with clamp is used to “coil” any extra cannula tubing length not needed and will keep the cannula tubing from sliding off as the tank is tilted to be pushed or being carried.

FIG. 10 illustrates a hook with a clip like that used with an earring to hold the cannula tube in place close to the tank.

FIGS. 11 and 12 illustrate a warning system for detecting low oxygen level in the tank or low oxygen flow according to the invention.

FIG. 13 illustrates an O₂ tank according to the invention.

FIG. 14 illustrates a customized oxygen tank holder attached to a wheelchair, walker, or backpack with a Velcro flap attached to the tank holder to provide storage for extra length of cannula tubing.

FIG. 15, which is FIG. 3 of U.S. Pat. No. 5,255,768 and illustrates a cord winding mechanism.

FIG. 16 illustrates a battery with a tab that is sealed over a cannula port such that when the tab is pulled out and the cannula port is attached to the oxygen source it activates a timer in a wireless device for a predetermined time period to alert the patient that it is time to replace the cannula.

FIGS. 17A and B illustrate a covering such as a sleeve around the tank according to the invention.

FIG. 18 illustrates a bar code on a cannula tube that may be scanned to activate a timer and an alert according to the invention.

FIG. 19 illustrates a warning system for detecting low oxygen level in the tank or low oxygen flow according to the invention.

FIG. 20 illustrates a retractable cannula tube connected to a tank having a button used to extend or release the cannula tube

FIG. 21A-21D illustrate another embodiment of a mechanism to attach to the cannula tube and to the patients clothing.

### A DETAILED DESCRIPTION OF THE INVENTION

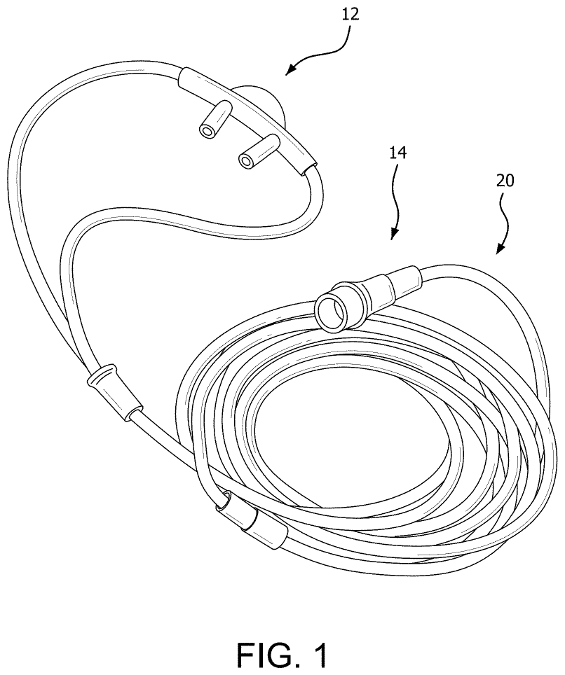

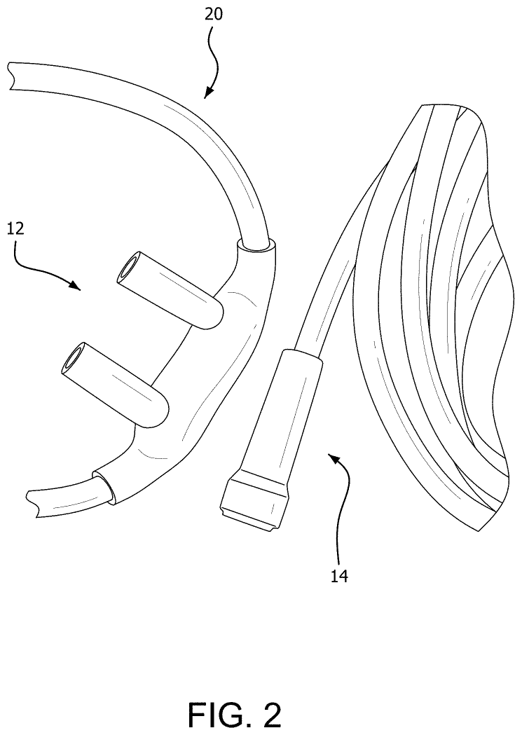

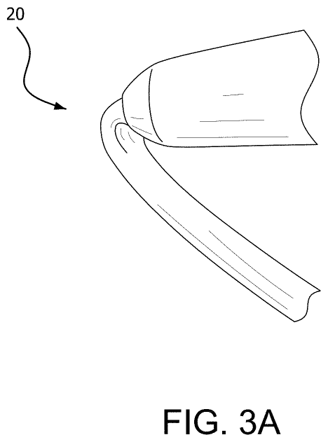

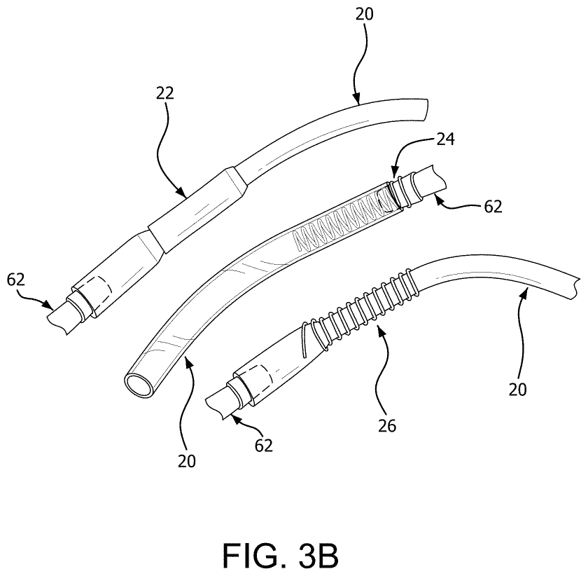

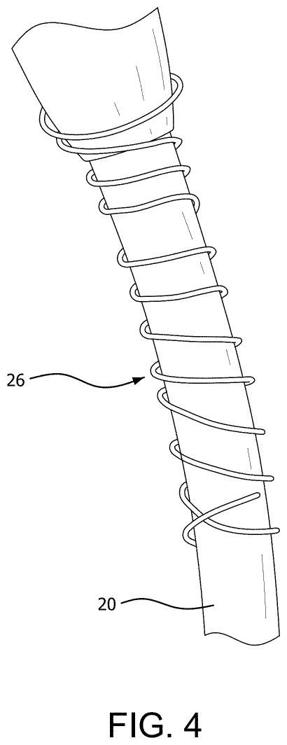

As shown in FIGS. 1 and 2, a cannula tube 20 has at one end a connector 14 that is used to connect to a regulator and an oxygen tank and a connector 12 that is used to connect a nasal cannula or nasal mask. An embodiment of the invention is to an improved structure and design for a nasal cannula, or a nasal mask cannula, and in particular to provide a structure or device interposed at the location point between the end of the cannula and its connection to the regulator or the oxygen supply tank 14, concentrator or supply to prevent crimping of the tube 20. FIG. 3A shows the cannula tube 20 according to the prior art, being crimped. As stated above, an object of the invention is to avoid crimping, and this may be accomplished as shown in FIG. 3B by providing a connecting means that prevents the cannula tube 20 to crimp. The regulator, concentrator or supply can be attached to the cannula by an anti-crimping means 22, 24, 26 which can be by using a longer tapered down tube 22 at the point of its entry and attachment to the inlet 62 from the oxygen supply system. Another anti-crimping means could be by providing an external coil like structure 26 or internal coils like structure 24 outside or within the cannula tubing 20 to avoid the crimping. FIG. 4 illustrates the external coil 26 in more detail. The use of a longer tapered down feed adapter 22, an internal coil 24 or external coil 26 prevents the cannula tube 20 from crimping at the end of the tube where it connects with the supply tank inlet 62.

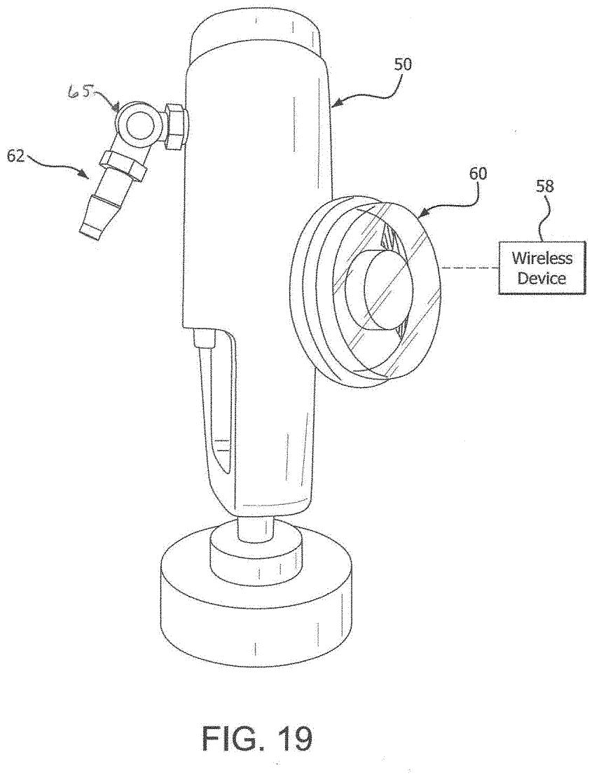

Another embodiment of this invention is to provide an improved regulator which includes an external outlet, port or coupling piece, where the regulator connects to the cannula, that incorporates a swivel pivot of at least 180° (and optimally 360°), or other device which prevents crimping of the cannula. The swivel pivot 65 is shown in FIG. 19 connected to supply tank inlet 62.

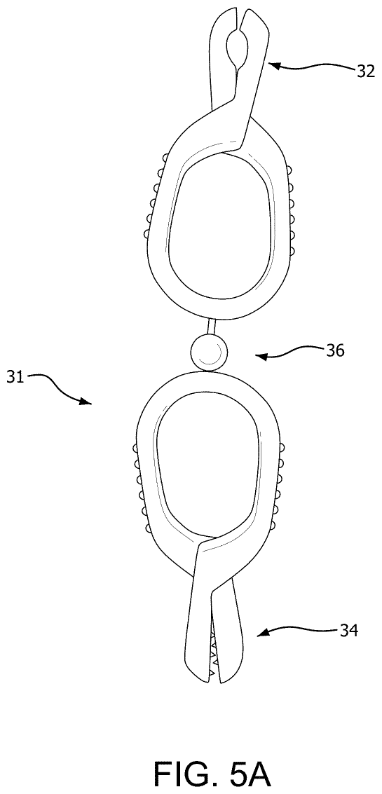

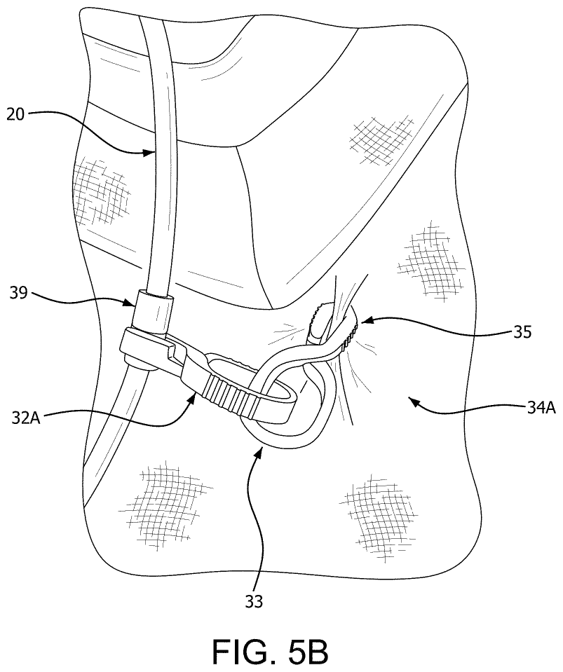

Another embodiment of this invention is to provide a clasp, clip or other device 32 or 32A which can be attached to a cannula as shown in FIGS. 5A and 5B that may be located on or attached to the cannula 20 below its bisection into two tubes 39 (as shown in FIG. 5B) such that the patient or a person can use the clasp, clip, or other device 34 or 34A to secure the cannula tubing or other conventional tubing to the patient's or person's clothing 35 to thereby reduce the tension or weight that the tubing exerts on the patient's ears and to keep the cannula close to the body. The appearance of the clasp, clip, or other device 31 or 33 may be stylized by the patient for personal preference. Again as shown in FIG. 5A, the connector 31 may comprise a dual-sided living hinge clip with a 360 degree pivot 36, with one end 34 attached to the patient's clothing 35 (shown in FIG. 5B) and the other end 32 attached to the cannula tube at the juncture where the cannula splits into two separate tubes to go around the patient's ears. The hinge clip 31 can be made of a metal or plastic, such as but not limited to nylon, polycarbonate, polyurethane, polyethylene or polypropylene. As shown in FIG. 5B, two integrated clasps 32A and 34A may be used without a 360 degree pivot 36.

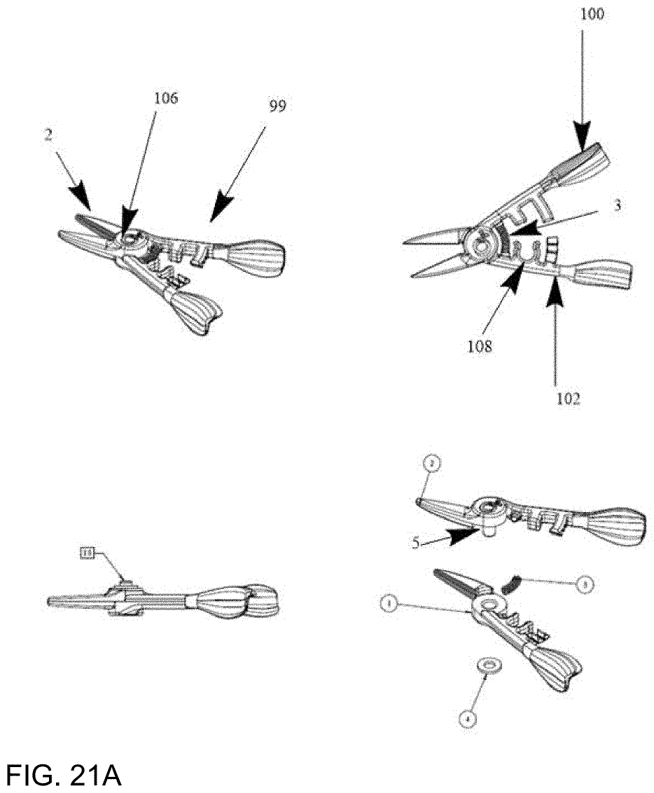

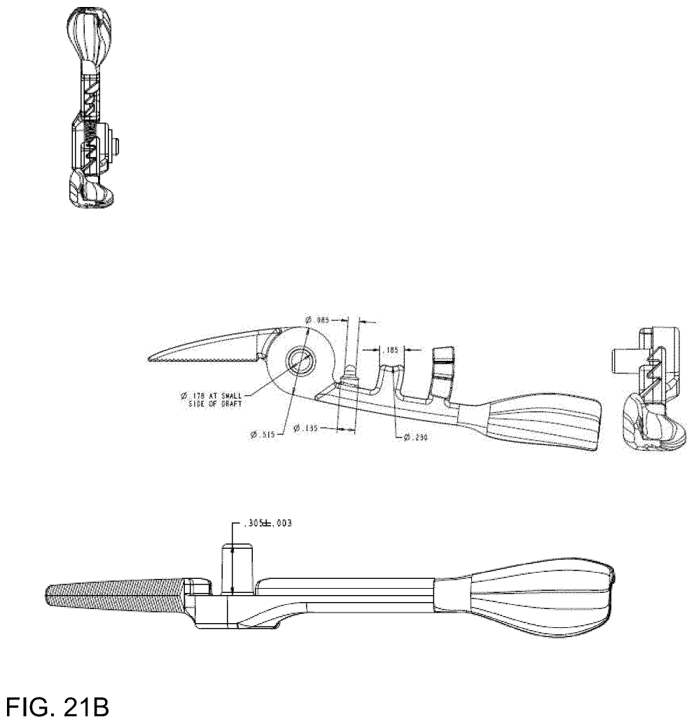

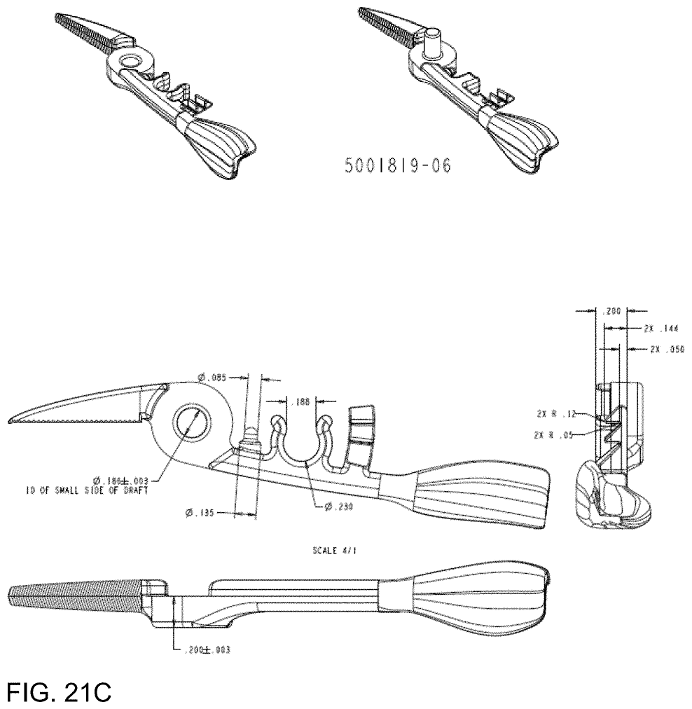

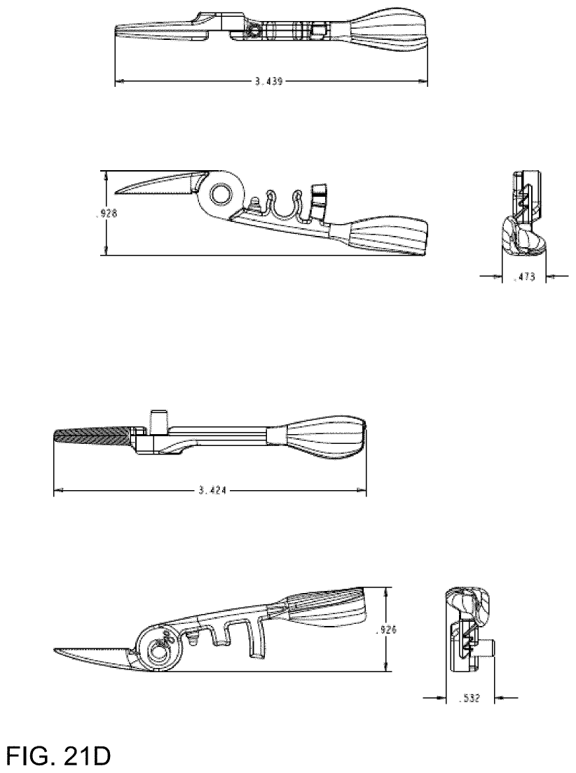

Another embodiment of this invention is illustrated in FIGS. 21A-D to provide a device 99 (clip or clasp) which can be attached to a cannula such that the patient can use the clasp 2 to secure the cannula to the patient's clothing to thereby reduce the tension or weight that the tubing exerts on the patient's ears and to keep the cannula close to the body to increase ease of movement and to reduce any tripping hazard. The patient use the device by squeezing the handles 100 and 102 and attach the end 2 to the patient's clothing to keep the tubing in place. One handle can incorporate a ‘u-shaped’ component to allow a person to snap the tubing in place within the u-shape component to secure the tubing in place. The u-shape component can be made to lock the tubing in place or to secure the tubing but still allow it to slide up and down through the u-shape. The u-shaped component should be made with sufficient flexibility and contact force such that the tubing is kept secured but will not kink or crimp when it is moved, bended, or twisted.

The patient's clothing includes anything the patient is wearing such as shirts, pants, hats, belt, etc. The device 99 could be made of two handle pieces 100 and 102 shown in FIG. 21A, and can be connected by a pin 5 built on handle 100 and washer 4 that attaches to handle 102 as shown as 106. To make the device move easier a spring 3 can be attached to the device 100 and 102 as shown in FIG. 21A. This would provide tension so that it is easier to open and close the device 99 by moving the handles 100 and 102. The cannula tube would be inserted in the gap 108 in the device 99. The cannula tubing would be able to be set in place in the device 99. The cannula tubing would be able to either be locked into place or move through the gap 108.

Again, the device 99 has one end capable of attaching to a person's clothing and at the other end a mechanism to open and shut the said one end capable of attaching to a person's clothing. FIGS. 21A-D show a more detail description of the device 99 with measurements in inches.

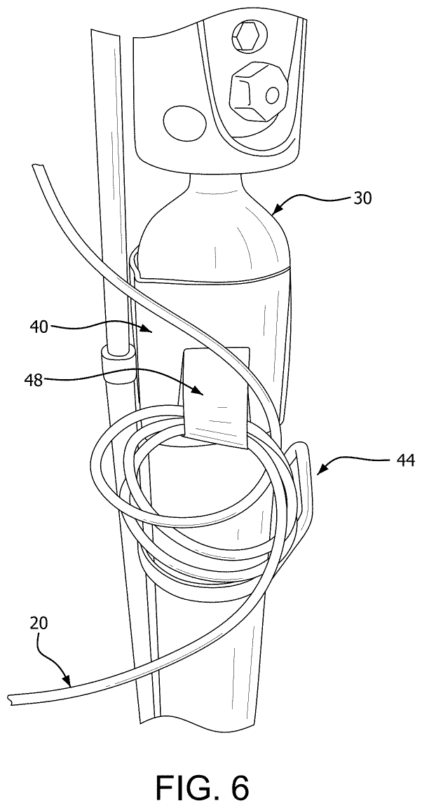

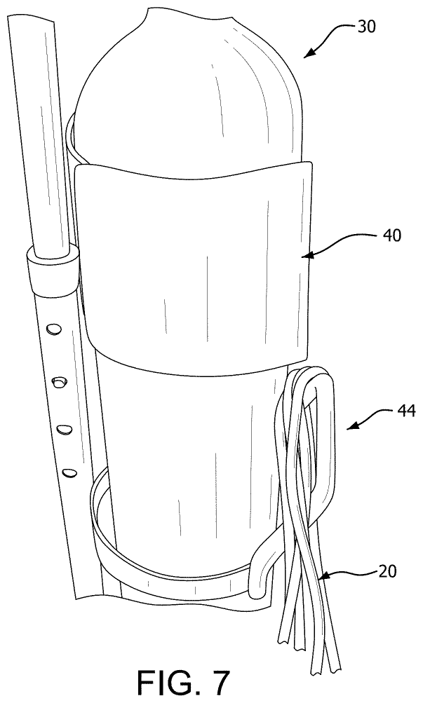

As shown in FIGS. 6 and 7, the structure can include an oxygen tank 30, and a storage or holding means 48 to hold or store un-needed length of cannula tubing 20. The holding means 48 can be, but is not limited to a Velcro® flap 48 or similar structure for winding up and holding a particular length of cannula tubing 20. Another holding means can be a hook 44 so that the cannula tubing is wrapped around the hook 44. The system shows an O₂ tank 30 with sock 40 and Velcro® flap to hold cannula tube 20. The sock 40 could also be manufactured to fit over top of the tank. The sock 40 could be different in color so as to distinguish the tank from another tank as discussed below.

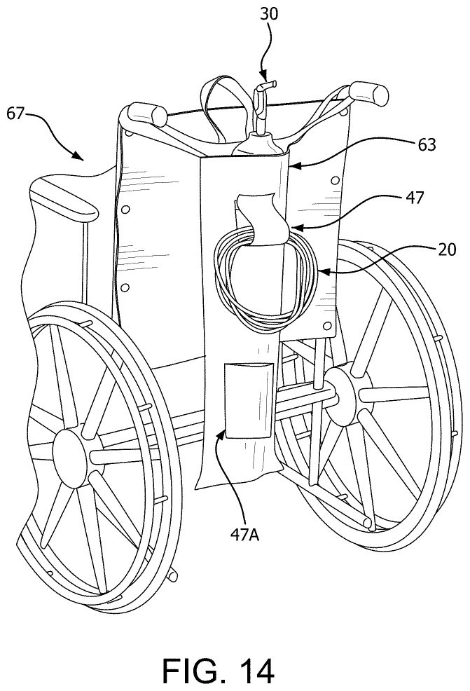

A further example is to have the oxygen tank 30 stored in a tank holder 63 as shown in FIG. 14. The oxygen tank 30 is shown in connection with a wheelchair 67. The tank holder 63 can be a sleeve with the bottom being sealed and the top being open to allow the oxygen tank 30 to be stored in the tank holder 63. The tank holder 63 can be made to be different than other tank holders by changing the standard tank holder, by color, size or an imprint as discussed below. The tank holder 63 can also include a storage or holding means 47 for storing or holding extra length of tubing. As shown in FIG. 14 the storage or holding means for the extra tubing can be a Velcro® flap or pocket. A second storage or holding means 47A can be present to store personal items, such as keys, credit cards, cell phones etc.

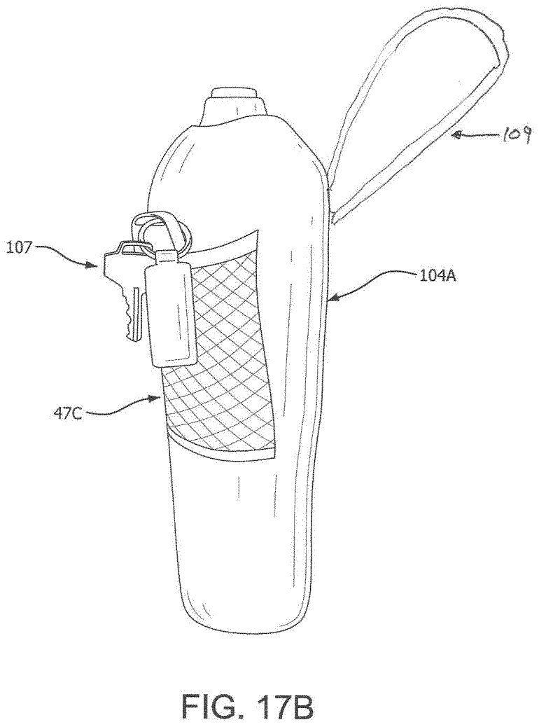

FIG. 17A illustrates another embodiment where a holding means 47B is attached to a tank holder sleeve 104 and/or a pocket or pouch that can store the cannula tube 20.

FIG. 17B illustrates a further embodiment where holding means 47C attached to a tank holder sleeve 104A that can store personal items 107 such as keys. The holder sleeve 104A can be a pocket. The holder sleeve 104A can be made of a transparent material so that you can see the items in the pocket or an opaque material so that you do not see the items in the pocket. There can be a shoulder strap 109 attached so that the tank can be easily be transported or carried. The strap 109 can be a conventional shoulder strap and can be adjustable.

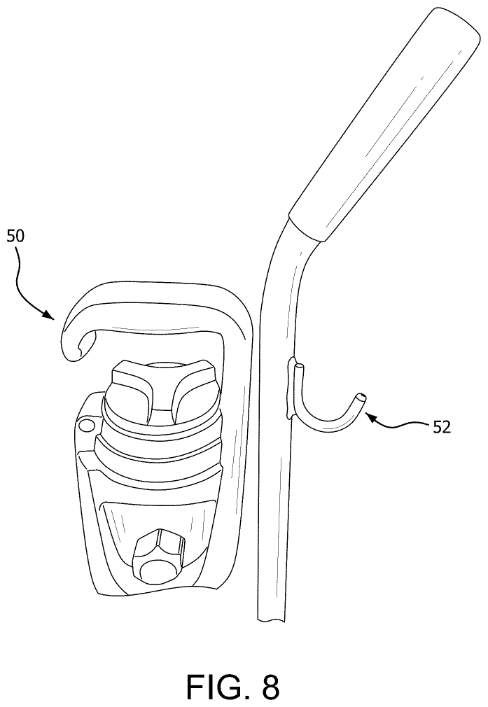

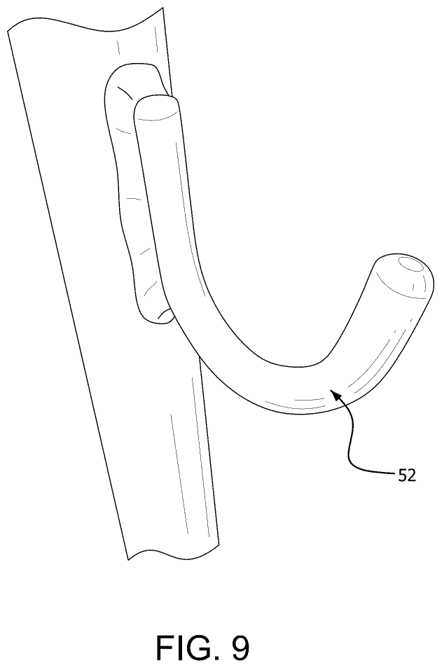

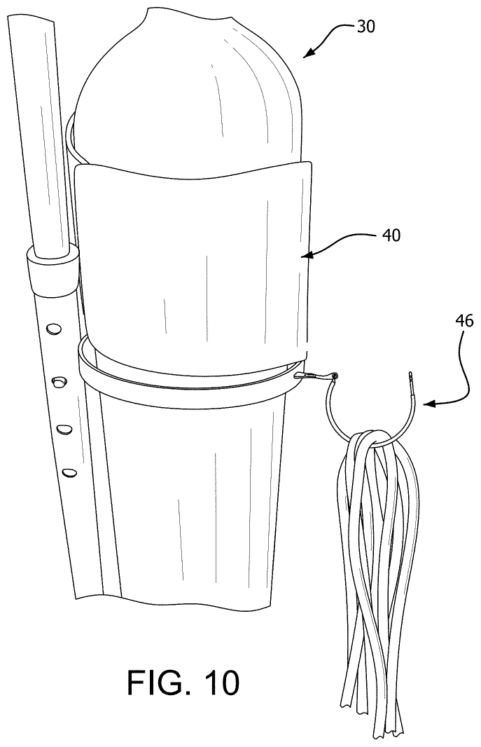

Another example is the use of a hook 52 as shown in FIGS. 8 and 9 that is attached to the oxygen tank 50 or cart and can be located in another area or same area on the cart. Another example is to use a clip attached to the tank holder as shown in FIG. 10 that may be opened and closed like that of an earring clip to hold the coil of extra unused cannula on the hook until needed. Examples include a hook 52 with a clip 46 like that used with an earring as shown in FIG. 10. The hook 52 on tank with clamp is used to coil extra cannula tubing and will keep extra tubing from sliding off as the tank is tilted to be pushed or carried.

FIGS. 6 and 7 illustrate one example comprising a tube-sock or cap-like structure 40 that can be placed over the top of or around the oxygen tank 30 which structure includes at least one opening through which the cannula tube 20 can be threaded and thereby secured to the oxygen tank 30, so as to provide storage for extra cannula length.

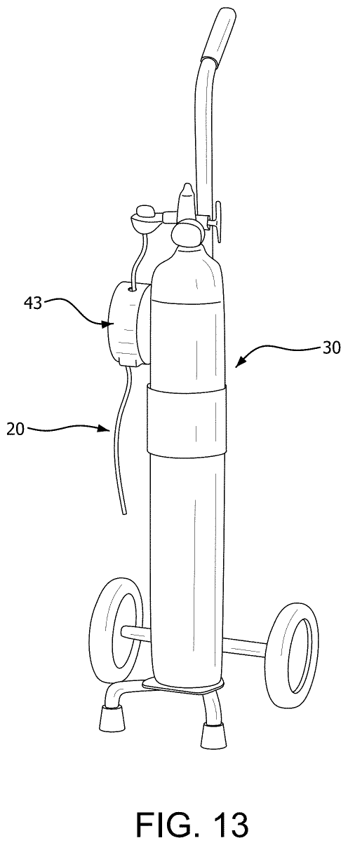

Another embodiment as shown in FIG. 13 is an improved design and structure by providing a coiling device or housing 43 which will house the tubing 20 such that the length of tubing is extended when the patient moves away from the oxygen tank 30 and needs additional length of tubing and retracts or contracts when the patient moves toward the oxygen tank and requires less length of tubing, which structure does not restrict the flow of oxygen. The coiling device or housing 43 can be light weight and easy to carry. The coiling device or housing 43 can also be of varying colors or display various logos such as the logo of a sports team or school so to distinguish the coiling device or housing 43 from other devices. This is discussed in more detail below.

The structure can further include an attachment that recoils and releases the cannula tube 20 length as needed. The attachment is used to retract the cannula tube 20 back to a device when it is pulled a certain distance from the device. The mechanism can be the same as used in the form of an electrical cord that retracts back to an appliance such as an iron or a vacuum cleaner. Included in the design of a device with a retractable cannula tube 20 can be a space for storage of the excess cannula tube 20 length. A retractable cannula tube 20 can work by providing a small storage space for excess cannula tube 20. The cannula tube 20 can be on a set of rotatable wheels which contain space for the entire length of the cannula tube 20. When the cannula tube 20 is extended outside of the device and reaches its maximum length, the user will feel tension on the cannula tube 20 and will not be able to move the device further without moving the device closer to the work area. The entire process is similar to the winding of a fishing wheel. A retractable cannula tube 20 can be on a set of rotating wheels; these wheels contain enough room for the entire length of the cannula tube 20. They also can have springs and ratchets that help lock the wheels when you pull out the cannula tube 20; this is why the cannula tube 20 doesn't snap back automatically when you try to extend it.

Instead, the cannula tube 20 may retract a tiny bit as the lock sets. When it's time to roll the cannula tube 20 back up, a patient usually would have to press a button to disengage the lock, and the wheels will turn until either the plug on the cannula tube 20 blocks further movement, or you give the cannula tube 20 another small tug to engage the lock.

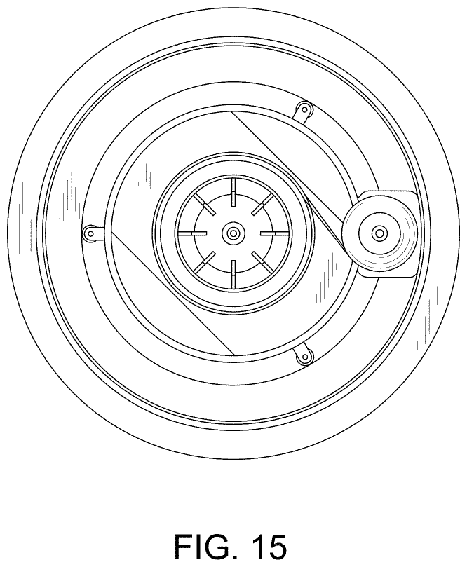

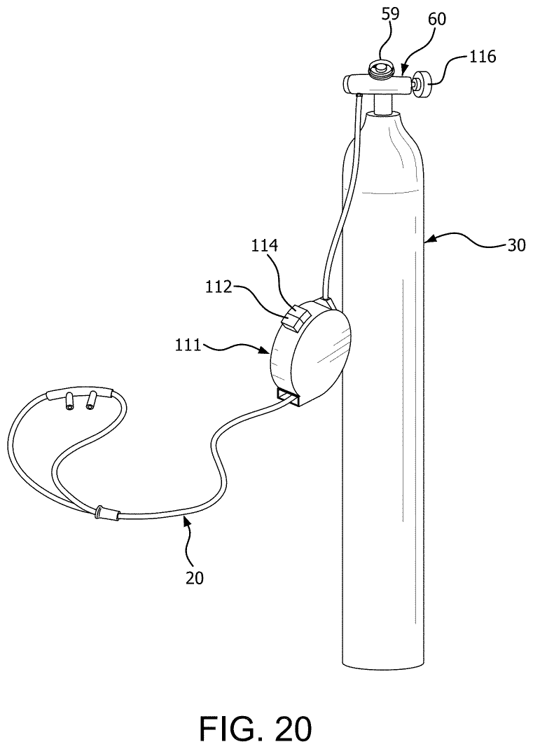

U.S. Pat. No. 5,255,768 entitled “Cord Winder Apparatus for a Vacuum Cleaner System” issued Oct. 26, 1993 is incorporated by reference in its entirety including the figures, especially for showing how a retractable cord system works which includes FIG. 3. FIG. 15 which is similar FIG. 3 of U.S. Pat. No. 5,255,768 illustrates a cord winding mechanism that could be used to wind the cannula tube 20 as described in U.S. Pat. No. 5,255,768. The structure could alternatively include a retractable mechanism such as a canister with a coiling apparatus inside, such as the mechanism used with some retractable dog leashes and tape measures, and garden hoses, which allows for the cannula tubing to be extended from the canister by pressing a button to provide extra length of cannula to permit the patient to move away from the tank and can be retracted into the canister by pressing the same or a different button when less length of cannula is needed such as when the patient moves closer to the tank. FIG. 20 shows a canister 111 holding the cannula tube 20. The canister 111 has a retractable mechanism with two buttons 112 and 114 provided. When the first button 112 is depressed the cannula tubing 20 is extended and the movement stops when the button 112 is released. When the second button 114 is pressed the cannula tubing 20 retracts and the movement stops when the button is release. It is noted that the knob 116 on the regulator 60 can be used to set the pressure of the O2.

Another possibility is the button 112 is pushed and the cannula tubing 20 is extended and the movement stops when the button 112 is released. To retract the cannula tubing 20, the cannula tubing 20 gets a small tug outwardly to disengage the lock and the cannula tubing is thereby retracted into the canister 111.

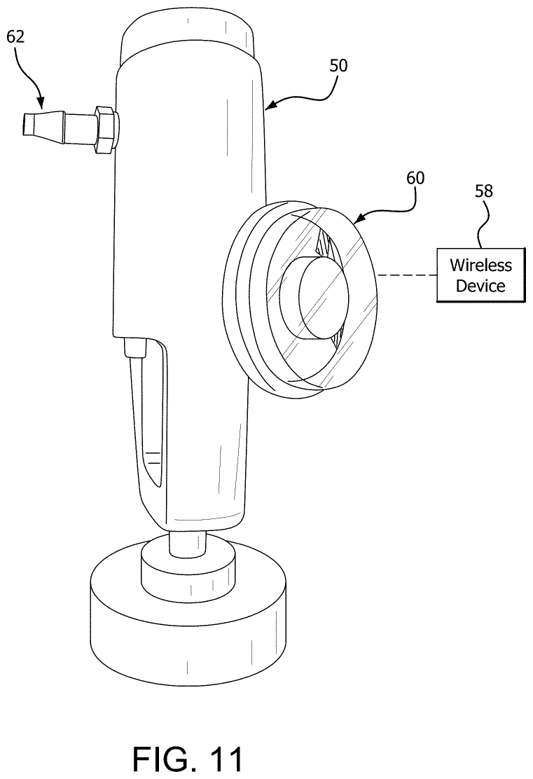

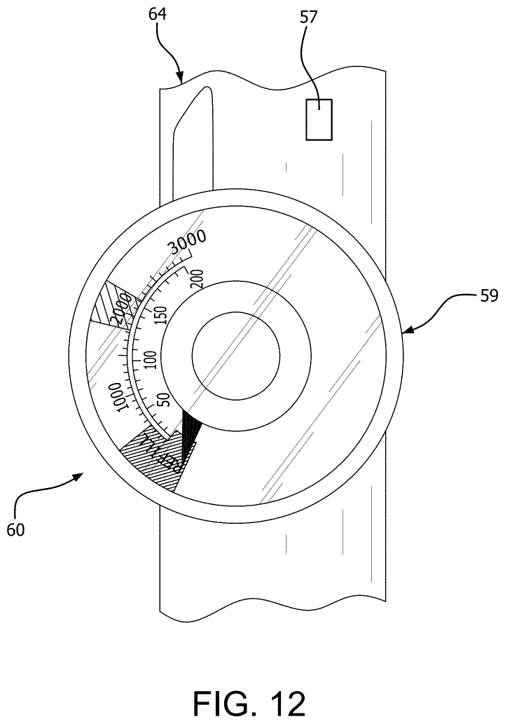

Another embodiment of the invention is described in FIGS. 11 and 12.

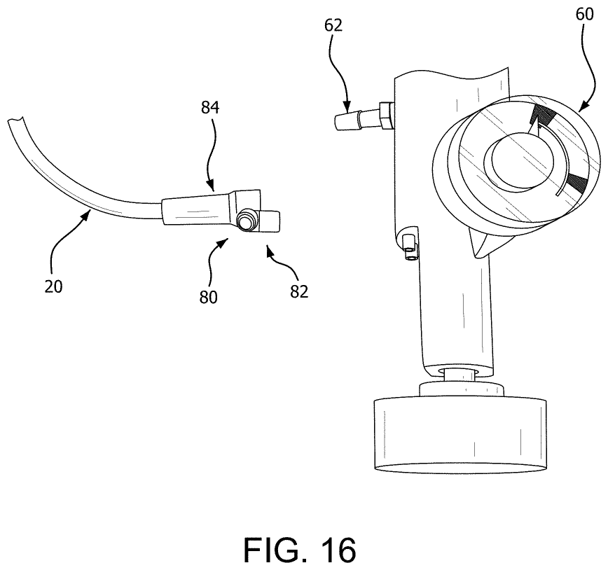

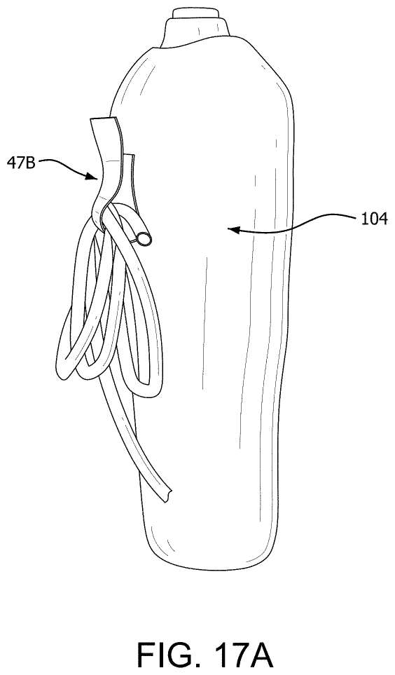

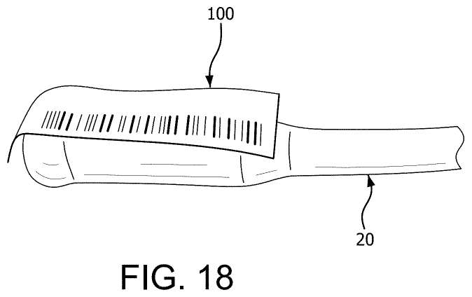

A regulator sensor 57 shown in FIGS. 11, 12 and 19 incorporated with the regulator 60 that detects the oxygen levels in pressure (converted into liters) that are currently available in the tank. The regulator 60's display 59 maybe a dial display as shown in FIGS. 11 and 12 or digital display. This sensor may be powered by a battery, like a 9 volt or AA battery. It may also provide a signal that alerts the patient when the oxygen level or pressure is lower than as set and a signal to alert the patient that the battery is low. This could be located at the regulator's inlet 64 from the tank just before the first reduction stage measuring raw pressure points. This may have adjustable or non-adjustable settings for the purpose of alerting the patient when oxygen delivery levels are reaching a low critical level such that a new source of oxygen is needed. This may also alert the patient of what level of oxygen flow the patient's oxygen supply is currently providing, and how much time the oxygen supply will continue to provide oxygen at that level. The sensor may interface with a wireless device so that a visual, audible, or vibration alerts may be provided by smartphone, smartwatch, or other wireless device 58 to warn the patient. Alternatively, the signal may be in the form of a light or vibration such as a flashing light that may be directed in multiple directions, iE ceiling, in front of them at eye sight levels both standing, sitting or lying down are a few examples. FIG. 19 illustrates a supply tank inlet 62 that incorporates a swivel pivot 65 of at least 180° (and optimally 360°), which prevents crimping of the cannula. A further embodiment of the invention is to provide a regulator 60 incorporating a mechanism or device 61 that provides a timing function and an interface with a digital device, such as a smartphone, a smartwatch, or other wireless device 58, such as an Apple watch or Fitbit, which timer starts when the cannula is connected to an oxygen tank and activated and notifies the patient when a pre-set time period such as two weeks (or other pre-set time) has expired, thus prompting the patient to replace the cannula tube 20 so as to avoid the deleterious effects of bacteria build up and tube hardening or deterioration. A device, mechanism, or structure 58 to provide the patient with a signal to alert when it is time to replace the cannula tube 20 which comprises a patient attaching a cannula tube to an oxygen source which connection sets a timing function, such as a timer on a smartphone or smartwatch for a pre-set time period such as two weeks, which will provide the patient a signal when the cannula is connected to the oxygen tank and activated. When the signal or alarm goes off from the timer, the patient knows to replace the cannula tube. For example, as shown in FIG. 16, a battery 80 (like a hearing aid battery, such as a 1.45 volt battery) that has a tab 82 release to start the timing function may be sealed over the cannula port 84 that attaches to the regulator or oxygen supply, whether tank, or concentrator. To attach the cannula tube 20 and its port 84 to the oxygen source, the tab release on the battery must be pulled off, activating a sensor that interfaces with a timer on a wireless device. Thus a 2 week (or other time period) timer will be set. Alternatively, as shown in FIG. 18, a bar code 100 could be added to the cannula tube 20 that when scanned starts a timer and provides an alert from a wireless device such as a smart phone after a predetermined time interval has passed.

Another embodiment of this invention to provide an improved nasal prong or nasal mask cannula, regulator and oxygen tank by providing a means of differentiating a patient's equipment from others and to permit the patient to stylize his or her equipment. This can be done in a variety of ways. For example, any of the following ways:
a cap or tapered sock can be fitted over the top of the oxygen tank with the cap or sock having an opening clasp, clip, or other device to hold extra length of cannula and being of varying colors or displaying various logos such as the logo of a sports team or school.
may incorporate a flap and fastening mechanism to allow a person to coil up and store unused length of tubing and secure it to the tank glove with the flap.
the cannula clasp or clip attached by a clip to the sliding collar at the bifurcation of the cannula into two ear sections below the patient's chin making the clasp or clips of varying colors or display various logos such as the logo of a sports team or school.
making the tank holder different from the standard tank holder, by color, size or an imprint.
altering the length of the cannula tube by having a storage system.

Another embodiment of the invention is a covering or device for the tank, such as a “sock” or “cap” or “sleeve,” or “glove” over or around the tank with one or more “pockets” or “pouches” which may be open or capable of being closed and optimally can provide for storage of a patient's personal items such as but not limited to a phone, wallet, keys, identification, credit cards, medicine, currency, etc.

Another embodiment is a cannula with an embedded bar code or a battery tab release on the cannula that when scanned or activated sets a timer for two weeks or other prescribed time period and provides an alert to the patient at the end of two weeks when the cannula should be changed.

The further embodiments include

A device which comprises a cannula tubing, a regulator, and an oxygen supply tank having a port base or a concentrator and an anti-crimping component is added at the port base of the oxygen tank or concentrator to prevent the cannula tube from crimping.

The device of embodiment 1, wherein there is included a longer tapered down tubing at the location of its entry point and attachment to the regulator of the oxygen supply tank.

The device of embodiments 1 or 2, wherein the connector is an internal coil, or external coil which prevents the cannula tube from crimping at the end of the tube.

The device of embodiments 1, 2 or 3, which further comprises a housing, nasal prongs or a nasal mask, connected to one end of the cannula tubing.

A device which comprises a housing, cannula tubing, nasal prongs or a nasal mask an oxygen supply tank or concentrator, and a regulator, wherein the cannula tube is attached in the housing with one end of the cannula tube being connected to the oxygen tank and the other end of the cannula tube being connected to nasal prongs or a nasal mask wherein the cannula tube is attached to the housing such that the length of tubing may be extended when the patient needs additional length of tubing and retracts or contracts when the patient moves toward the oxygen tank and requires less length of tubing, which structure does not crimp or otherwise restrict the flow of oxygen.

The device of embodiment 5, wherein the cannula tube is connected by means of a vacuum cleaner-like coiling mechanism.

The device of embodiment 5 where the cannula tubing is connected to a retractable coiling mechanism which includes one or more buttons to extend or retract the cannula like that used in some retractable dog leashes, tape measures, or garden hoses.

A device comprising a housing, cannula tubing, nasal prongs or a nasal mask, an oxygen supply tank or concentrator, and a regulator, and a device such as one or two clasps or clips placed on the cannula below the point where it separates into two separate tubes at the sliding of the collar which clasp or clip may be attached to the patient's clothing to relieve the tension or weight exerted on the patient's ears by the cannula.

The device of embodiment 7, further comprising a dual-sided hinged clip wherein the cannula tubing is attached to the dual-sided hinged clip with a 360 degree pivot.

A device which comprises a housing, cannula tubing, nasal prongs or nasal mask, an oxygen supply tank or concentrator, and a regulator which includes a storage device or mechanism for holding extra length of the cannula tubing when not needed by the patient.

The device of embodiment 9 wherein a cap, glove, sleeve, or sock with an opening is placed around or over the top of the oxygen tank through which the cannula may be threaded and wherein the cap, glove, sleeve, or sock utilizes a flap, hook, loop, or clasp to hold or store excess cannula tubing.

A device comprising a housing, cannula tubing, nasal prongs or a nasal mask, an oxygen supply tank or concentrator, and a regulator which includes a sensor or other warning mechanism to detect when oxygen level or oxygen pressure is low and to provide a warning or alarm to the patient.

The device of embodiment 11 wherein the warning sensor includes an interface with a wireless device like a smartphone or smartwatch which emits a visual or audible alarm or vibration alert.

A device which comprises a housing, cannula tubing, nasal prongs or a nasal mask, an oxygen supply tank or concentrator, and a regulator which includes a timing device or mechanism wherein the patient may set the timing mechanism for a preset time period, which will alert the patient with visual or audial or vibration alert after the preset time period has expired to replace the cannula so as to avoid the deleterious effects of the bacteria buildup and hardening or deterioration of the cannula.

The device of embodiment 13, wherein the cannula system includes a bar code, tab release, or a sensor at the end of the cannula where the cannula connects to the regulator which may be scanned or activated when the cannula is first put into use and which starts a timer of a prescribed time period such as two weeks duration and provides a color change or an audio, visual, or vibration alert to the patient when the time period has passed and the cannula should be changed.

A device of embodiments 13 or 14, wherein the cannula system includes a battery with a tab between the battery and the cannula at the location where it is connected to the oxygen source, such that the tab on the battery must be pulled off to activate a timer and to unseal the opening to the battery so as to be able to slide it over the tank's oxygen exit port.

A process of providing a means of differentiating a patient's equipment from another patient's equipment wherein the equipment comprises an oxygen tank, tank holder, a cannula tube and nasal prongs on a nasal mask and optionally a cannula clip or clasp wherein the process comprises at least one of the following:
A. making the cannula tube of varying colors, or displaying various logos;
B. making the tank holder look different from the standard tank holder; and
C. if a cannula clasp or clip fitted at the bifurcation of the cannula into two ear sections below the patient's chin is present, making the clasp or clips of varying colors or display various logos.

A covering, preferably a “cap”, “sock”, “sleeve,” or “glove” which may be placed over or around a gas supply tank or concentrator that is used with a regulator to supply gas like oxygen to a patient.

The covering of embodiment 17, wherein the covering is made of neoprene or similar fabric that makes the tank easier to handle and that provides a cushion when two or more metal tanks are stacked together.

The covering of embodiment 17 or 18, wherein the sock, cap, sleeve, or glove further includes one or more open or closed pockets or pouches for storing a patient's personal items such as a phone, wallet, keys, identification, currency, or other items.

The covering of embodiments 17, 18 or 19, wherein the sock, cap, sleeve, or glove further includes a handle or grip to carry the gas tank.

A device comprising a gas supply tank or a concentrator that is used with a regulator to supply gas like oxygen to a patient and a covering wherein the covering is placed over or around the gas supply tank or the concentrator.

The device of embodiment 21, wherein the covering is made of neoprene or similar fabric that makes the tank easier to handle and that provides a cushion when two or more metal tanks are stacked together.

The covering of embodiment 21 or 22, wherein the covering is a sock, cap, sleeve, or glove further includes one or more open or closed pockets or pouches for storing a patient's personal items such as a phone, wallet, keys, identification, currency, or other items.

The covering of embodiments 21, 22 or 23, wherein the covering is a sock, cap, sleeve, or glove further includes a handle or grip to carry the gas tank.

The device of embodiments 1-15, wherein the regulator comprises an external port or connector piece and a swivel pivot that can rotate at least 180° and optimally 360°, and said external port or connector piece is attached to the swivel pivot and connects to the cannula tube such that the swivel pivot and rotate and prevents or reduces crimping of the cannula tube.

In summary, the invention relates to a nasal prong or nasal mask cannula gas supply system which has at least one of the following features: to enhance the cannula tubing at the port base of the oxygen source to minimize “crimping” and diminished oxygen delivery; to design a cannula that will expand in length as needed and shorten as needed without loss of pressure (PSI) of oxygen from start point of oxygen source to patient; to provide a device such as a clasp or clip attached to the cannula which clasp or clip may be attached to the patient's clothing to relieve pressure or weight exerted by the cannula tubing on the patient's ears and keep the cannula close to the patient's body; to provide a storage means for holding extra length of cannula tubing, like a “cap,” “sleeve,” “glove,” or “sock” fitted to cover the top of the oxygen tank, which includes an opening, loop, clip, clasp or other device through which the cannula tubing may be threaded to hold extra cannula length to be held out of the patient's way and not crimp; to provide an open or closed covering over or around the gas tank that may include pockets or pouches that are open or capable of being closed for storage of personal items; to provide a warning sensor or other device to alert the patient that the level of oxygen in the oxygen supply is low or the flow rate is above or below the prescribed flow rate; to provide a timing device or mechanism to alert the patient that a preset time period, such as two weeks, has expired since the time of the cannula's first use so the patient knows it is time to replace the cannula tubing to avoid the deleterious effects of the build up of bacteria or hardening or deterioration of the tubing; to provide ways to “personalize” the cannula with color and/or design making it easier to find their tank, oxygen portable case or wheel chair case; to provide an oxygen supply system whereby the regulator comprises an external outlet or port for connection to the cannula which includes a pivot of 180° and optimally 360° to prevent crimping of the cannula.

We claim:

1. A device which comprises a cannula tubing, a regulator, and an oxygen supply tank having a port base or a concentrator, nasal prongs or a nasal mask and an anti-crimping component which is selected from the group consisting of
a) an external coil which located on the cannula tubing at the end connected to the port base of the oxygen tank or concentrator to prevent the cannula tube from crimping and
b) an internal coil which is located only partially in the cannula tubing at the end connected to the port base of the oxygen tank or concentrator to prevent the cannula tube from crimping and wherein the cannula tubing is continuous having one end connected to the port base of the oxygen tank or concentrator and the other end connected to nasal prongs or a nasal mask.

2. The device as claimed in claim 1, wherein there is included a longer tapered down tubing at the location of its entry point and attachment to the regulator of the oxygen supply tank.

3. The device as claimed in claim 1, wherein the anti-crimping component is an external coil.

4. A clip or clasp which is capable of being connected to a cannula tubing or other tubing or cords wherein the tubing or cords is capable of sliding in the clip or clasp and will not crimp and the device can also be attached to a person's clothing, garment, or hat to keep the tubing or cords in place.

5. A device which comprises a housing, cannula tubing, nasal prongs or a nasal mask, an oxygen supply tank or concentrator having an inlet, anti-crimping component and an oxygen regulator, and said anti-crimping component which is selected from the group consisting of
a) a tapered down tubing located at the entry point and attachment to the regulator of the oxygen supply tank or concentrator inlet to prevent the cannula tube from crimping,
b) an external coil which located on the cannula tubing at the end connected to the oxygen tank or concentrator inlet to prevent the cannula tube from crimping and
c) an internal coil which is located only partially in the cannula tubing at the end connected to the supply tank or concentrator inlet to prevent crimping of the cannula tube and
wherein the cannula tube is attached in the housing with one end of the cannula tube being connected to the oxygen regulator or oxygen supply tank inlet and the other end of the cannula tube being connected to nasal prongs or a nasal mask wherein the cannula tube is attached to the housing such that the length of tubing may be extended when the patient needs additional length of tubing and retracts or contracts when the patient moves toward the oxygen tank and requires less length of tubing, which structure does not crimp the cannula or otherwise restrict the flow of oxygen.

6. The device as claimed in claim 5, wherein the cannula tube is connected by means of a vacuum cleaner-like coiling mechanism.

7. The device as claimed in claim 5, where the cannula tubing is connected to a retractable coiling mechanism which includes one or more buttons to extend or retract the cannula like that used in some retractable dog leashes, tape measures, or garden hoses.

8. A device comprising a housing, cannula tubing, nasal prongs or a nasal mask, an oxygen supply tank or concentrator, and a regulator, and a device such as one or two clasps or clips placed on the cannula below the point where it separates into two separate tubes at the sliding of the collar which clasp or clip may be attached to the patient's clothing to relieve the tension or weight exerted on the patient's ears by the cannula.

9. The device as claimed in claim 5, further comprising a dual-sided hinged clip wherein the cannula tubing is attached to the dual-sided hinged clip with a 360 degree pivot.

10. A device which comprises a housing, cannula tubing, nasal prongs or nasal mask, an oxygen supply tank or concentrator, and a regulator which includes a storage device or mechanism for holding extra length of the cannula tubing when not needed by the patient.

11. The device as claimed in claim 10, wherein a cap, glove, sleeve, or sock with an opening is placed around or over the top of the oxygen tank through which the cannula may be threaded and wherein the cap or sock utilizes a flap, hook, loop, or clasp to hold or store excess cannula tubing.

12. A device comprising a housing, cannula tubing, nasal prongs or a nasal mask, an oxygen supply tank or concentrator, and a regulator which includes a sensor or other warning mechanism to detect when oxygen level or oxygen pressure is low and to provide a warning or alarm to the patient.

13. A device claimed in claim 12, wherein the sensor is placed within the nasal prongs or mask.

14. The device as claimed in claim 12, wherein the warning sensor includes an interface with a wireless device which emits a visual or audible alarm or vibration alert when the oxygen level or oxygen pressure is low.

15. A device which comprises a housing, cannula tubing, nasal prongs or a nasal mask, an oxygen supply tank or concentrator, and a regulator which includes a timing device or mechanism wherein the patient may set the timing mechanism for a preset time period, which will alert the patient after the preset time period has expired to replace the cannula so as to avoid the deleterious effects of the bacteria buildup and hardening or deterioration of the cannula with visual or audial or vibration alert.

16. The device as claimed in claim 15, wherein the cannula system includes a bar code or a sensor at the end of the cannula where the cannula connects to the regulator which may be scanned or activated when the cannula is first put into use and which starts a timer of a prescribed time period and provides a color change or an audio, visual, or vibration alert to the patient when the time period has passed and the cannula should be changed.

17. A device as claimed in claim 15, wherein the cannula system includes a battery with a tab between the battery and the cannula at the location where it is connected to the oxygen source, such that the tab on the battery may be pulled off to activate a timer and provide an audio, visual, or vibration alert to the patient after a prescribed time period such as two weeks.

18. A process of providing a means of differentiating a patient's equipment from another patient's equipment wherein the equipment comprises a tank holder and optionally a cannula clip or clasp and optionally a cannula tube wherein the process comprises at least one of the following:
A. making the tank holder look different from the standard tank holder;
B. if the cannula tube is present making the cannula tube of varying colors, or displaying various logos; and
C. if the cannula clasp or clip is present the cannula clasp or clip fitted at the bifurcation of the cannula into two ear sections below the patient's chin is present, making the clasp or clips of varying colors or display various logos.

19. A covering of selected color and design which may be placed over or around a gas supply tank or concentrator which differentiates the tank supply system from others and further comprises one or more open or closed pockets or pouches capable for storing a person's at least one personal item or tubing.

20. The covering as claim in claim 19, further comprises one or more open or closed pockets or pouches capable for storing a person's at least one personal item or tubing.

21. The covering as claim in claim 19, wherein the covering is made of neoprene or similar fabric that makes the tank easier to handle and that provides a cushion when two or more metal tanks are stacked together.

22. The covering as claim in claim 21, wherein the at least one personal item is selected from the group consisting of a phone, wallet, keys, money, pills and identification.

23. The covering as claim in claim 21, wherein the covering is a sock, cap, sleeve or glove and further includes a handle, shoulder strap or grip to carry the gas tank.

24. The covering as claim in claim 19, wherein the covering further incorporates a storage means for coiling up unused cannula tubing.

25. The covering as claim in claim 24, wherein the storage means is a flap with a fastening mechanism.

26. The device as claimed in claim 1, wherein the regulator comprises an external outlet, port or connector piece and a swivel pivot that can rotate at least 180° and optimally 360°, and said external outlet, port or connector piece is attached to the swivel pivot and connects to the cannula tube such that the swivel pivot rotates and prevents or reduces crimping of the cannula tube.

27. The device as claimed in claim 5, which further comprises one or two clasps or clips placed on the cannula below the point where it separates into two separate tubes at the sliding of the collar which clasp or clip may be attached to the patient's clothing to keep the cannula close to the patient's body and to relieve the tension or weight exerted on the patient's ears by the cannula.

28. The clip or clasp as claimed in claim 4, wherein the clip or clasp has one end capable of attaching to a person's clothing and at the other end a mechanism to open and shut the said one end capable of attaching to a person's clothing.

29. The clip or clasp as claimed in claim 28, which further comprises two handles with flat surfaces for placement of fingers when squeezing the handles and are connected by a pin or screw and washer attached to the handles.

30. The clip or clasp as claimed in claim 29, which further comprises a spring which is incorporated between the handles to provide tension and make it easier to open and close the clip.

31. The clip or clasp as claimed in claim 29, wherein said one handle incorporates a u-shaped component where the tubing or cord(s) can be placed and which keeps the tubing or cord(s) secured.

32. The clip or clasp as claimed in claim 31, wherein the u-shaped component keeps the tubing or cord(s) secure but allows the tubing or cord(s) to slide up and down within the u-shape.

33. A process which comprises attaching the clip or clasp of claim 4 to the tubing to keep the tubing close to a body of a person to improve ease of movement and to reduce the risk of tripping and to secure the tubing without crimping or kinking.
